(19) United States
(12) Patent Application Publication
(10) Pub. No.: US 2010/0260713 A1
(43) Pub. Date: Oct. 14, 2010

(54) Compositions and Methods for the Inhibition of Hepatitis C Viral Replication with Structural Analogs

(75) Inventors: GEORGE Y. WU, AVON, CT (US); CATHERINE H. WU, AVON, CT (US); ROBERT SMOLIC, NEW BRITAIN, CT (US); ROBERT SMITH, BRISTOL, CT (US)

Correspondence Address: JOHN S. PRATT, ESQ;KILPATRICK STOCKTON, LLP, 1100 PEACHTREE STREET, SUITE 2800, ATLANTA, GA, 30309, US

(73) Assignee: UNIVERSITY OF CONNECTICUT, FARMINGTON, CT (US)

(21) Appl. No.: 12/757,828

(22) Filed: Apr. 9, 2010

Related U.S. Application Data
(60) Provisional application No. 61/168,058, filed on Apr. 9, 2009.

(51) Int. Cl. A61K 31/7088; C07H 21/02; C12N 7/00; A61K 38/21; A61P 31/12; G06F 19/00

(52) U.S. Cl. 424/85.4; 536/23.1; 514/44 R; 435/235.1; 702/19

(57) ABSTRACT

Compositions and methods for the inhibition of viral replication are provided. In some embodiments, the compositions include a nucleic acid sequence that is identical to a region of the hepatitis C virus (HCV) genome. In other embodiments, the compositions may include a nucleic acid sequence that has at least about 45% to about 95% sequence identity to the native HCV sequence and that has a similar secondary and tertiary structure to the native HCV sequence. In other embodiments, the compositions may include a nucleic acid sequence that does not have significant sequence identity to the native HCV sequence and that has a similar secondary and tertiary structure to the native HCV sequence. Also provided are methods for the treatment of a patient having an HCV infection by administering one of the compositions described herein, and uses of the compositions described herein in the manufacture of a medicament for the inhibition of HCV replication.

### PRIOR RELATED APPLICATIONS

The present application claims the benefit of priority to U.S. Provisional Application No. 61/168,058 filed Apr. 9, 2009, which is hereby incorporated by reference in its entirety.

### STATEMENT REGARDING FEDERALLY SPONSORED RESEARCH

The present invention was made with United States government support under National Institutes of Health (NIH) Grant No. DK-74891 and under National Institute of Diabetes and Digestive and Kidney Diseases (NIDDK), Grant No. RO1-DK042182. Accordingly, the United States government has certain rights in the invention.

### FIELD OF THE INVENTION

This application relates to compositions and methods for the inhibition of hepatitis C virus (HCV) replication. In particular, the application provides novel nucleic acid sequences that have a structural, rather than a sequence-specific effect, pharmaceutical compositions comprising such sequences, and methods for using such sequences and compositions to inhibit HCV replication.

### BACKGROUND

Hepatitis C virus (HCV) is a small, enveloped, single strand RNA virus. Chronic HCV infection is a leading cause of chronic hepatitis, and its sequelae, liver cirrhosis and hepatocellular carcinoma. It has been estimated that over 170 million people worldwide have been infected by HCV. There is no vaccine for the prevention of HCV infection, and current therapeutic options are limited, often not effective, associated with significant adverse effects, and costly. Accordingly, there is strong impetus to develop novel therapeutic strategies for the treatment and prevention of HCV infection that act through different mechanisms.

HCV has a positive strand RNA genome that consists of a single open reading frame of approximately 9,600 bases. This RNA genome includes both 5′ and 3′ untranslated regions (UTRs) that are important for the efficient replication of the viral genome and for the translation of the viral proteins. The 5′ UTR includes an internal ribosomal entry site that initiates the translation of an approximately 3,000 amino acid polypeptide that is cleaved by cellular and viral proteases to produce 10 active proteins (i.e., three structural proteins and seven non-structural (NS) proteins). HCV replicates rapidly in the hepatocytes of the liver using the host cell's machinery, and can produce about one trillion particles per day in a single infected individual. RNA replication occurs via the HCV RNA-dependent RNA polymerase NS5B, which produces a negative strand RNA intermediate which serves as a template for the production of positive strand RNA strands. HCV has a significant mutation rate, and therefore, is able to respond to and evade the efforts of its host's immune system, making the task of treating HCV infection very challenging.

HCV isolates may be categorized based on their sequences into one of six genotypes (i.e., 1-6), which can then be further categorized into several different subtypes within a genotype (e.g., 1a, 1b). Different HCV genotypes and subtypes are predominant in different geographical areas. Infection with one HCV genotype does not provide immunity to the patient against HCV of that genotype or any other genotypes, and therefore, concurrent infection with more than one HCV genotype isolates is possible. In addition, because different HCV genotypes and subtypes respond differently to the currently available therapies, the sustained virological response rates for therapy vary substantially between HCV genotypes.

For example, most patients currently are treated with 48 weeks of treatment with pegylated interferon and the antiviral nucleoside analog drug ribavirin, resulting in only a 40-50% sustained viral response rate in patients infected with the most common HCV genotype in the United States. In certain U.S. subpopulations, such as African American, Latino, and obese patients, the response rates are even lower, in the 20-30% range. In addition to the limited success rates with this course of treatment, the agents used in that treatment also have considerable reversible and irreversible side effects.

Certain other strategies have been studied such as, for example, the use of small molecules that interact with limited regions of target HCV proteins (e.g., the polymerase or protease). These small molecules include, for example, antisense molecules, ribozymes, RNAi, and nucleoside analogs. However, while many of those agents have been shown to be very potent in terms of inhibiting viral replication, they are susceptible to the problems of viral RNA inaccessibility and the development of resistance of the virus due to its rapid mutation rate.

What is needed, therefore, are effective molecules, compositions, and methods for the treatment of HCV infection without significant adverse effects. In particular, compositions that inhibit viral replication of HCV genomes in a patient or animal and that are not susceptible to inactivation by the rapidly mutating HCV genome are needed. Furthermore, compositions and methods that are effective in inhibiting replication of the genomes of isolates of more than one HCV genotype or subtype are needed. Also needed are methods for identifying structural analogs of viral nucleic acids that inhibit viral replication.

### SUMMARY OF THE DISCLOSURE

There is a great need for compositions and methods for the treatment of HCV infection. Such compositions and methods are provided herein. The novel nucleic acids disclosed herein have a structural, rather than a sequence-specific effect, and therefore are capable of inhibiting replication of more than one HCV genotype or subtype.

Novel sequences and compositions for the treatment and prevention of HCV infection are provided. The present compositions include an isolated nucleic acid sequence that has a similar or identical secondary structure to a native NS5B, X, BA, or EC region of a hepatitis C viral RNA. In some embodiments, the isolated nucleic acid sequence has a similar or identical secondary structure to the native NS5B region of the hepatitis C viral RNA, but the nucleic acid sequence does not have significant sequence identity to the NS5B region. In certain embodiments, the isolated nucleic acid sequence binds to or inhibits the activity of NS5B polymerase. In some embodiments, the isolated nucleic acid sequence has a length of about 50 to about 150 bases. In one embodiment, the isolated nucleic acid sequence is about 95 bases. In certain embodiments, the isolated nucleic acid sequence differs from the native NS5B, X, BA, or EC region in a sequence which forms a stem of a stem-loop secondary structure.

Additional compositions are provided that include an isolated nucleic acid sequence selected from the group consisting of SEQ ID NO:3, SEQ ID NO:2, SEQ ID NO:5, SEQ ID NO:9, SEQ ID NO:14, SEQ ID NO:15, SEQ ID NO:16, SEQ ID NO:17, SEQ ID NO:18, SEQ ID NO:19, and SEQ ID NO:20. In one embodiment, the isolated nucleic acid sequence is SEQ ID NO:3.

Still additional compositions are provided that include an isolated nucleic acid sequence having between about 45% and about 95% sequence identity to a sequence selected from the group consisting of SEQ ID NO:1, SEQ ID NO:2, SEQ ID NO:4, SEQ ID NO:6, SEQ ID NO:8, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar or identical secondary structure to SEQ ID NO:1, SEQ ID NO:2, SEQ ID NO:4, SEQ ID NO:6, SEQ ID NO:8, SEQ ID NO:14, SEQ ID NO:15, SEQ ID NO:16, SEQ ID NO:17, SEQ ID NO:18, SEQ ID NO:19, or SEQ ID NO:20. In certain embodiments, the isolated nucleic acid sequence has at least about 46% sequence identity to the selected sequence. In one embodiment, the isolated nucleic acid sequence is SEQ ID NO:2. In certain embodiments, compositions are provided that include an isolated nucleic acid sequence having between about 45% and 73% sequence identity to a sequence selected from the group consisting of SEQ ID NO:3, SEQ ID NO:5, and SEQ ID NO:9, wherein the isolated nucleic acid sequence has a similar or identical secondary structure to SEQ ID NO:3, SEQ ID NO:5, or SEQ ID NO:9. In other embodiments, compositions are provided that include an isolated nucleic acid sequence having between about 75% and about 95% sequence identity to a sequence selected from the group consisting of SEQ ID NO:3, SEQ ID NO:5, and SEQ ID NO:9, wherein the isolated nucleic acid sequence has a similar or identical secondary structure to SEQ ID NO:3, SEQ ID NO:5, or SEQ ID NO:9. In certain embodiments, the isolated nucleic acid sequence differs from the selected sequence in a portion of the selected sequence which forms a stem of a stem-loop secondary structure.

Any of the present compositions may further include a pharmaceutically acceptable carrier. In certain embodiments, the compositions include at least two different isolated nucleic acid sequences, wherein each isolated nucleic acid sequence has a similar or identical secondary structure to a native NS5B, X, BA, or EC region of a hepatitis C viral RNA. Any of the present compositions may be used for the manufacture of a medicament for the treatment of hepatitis C infection.

Methods for inhibiting replication of a hepatitis C virus are provided, which include adding a composition to the virus, wherein the composition includes an isolated nucleic acid sequence that has a similar or identical secondary structure to a native NS5B, X, BA, or EC region of a hepatitis C viral RNA and may or may not have significant sequence identity to the native region. In one embodiment, the nucleic acid sequence has a similar or identical predicted secondary structure to a native NS5B region, but does not have significant sequence identity to the NS5B region. In other embodiments, the methods for inhibiting replication of a hepatitis C virus include adding a composition to the virus, wherein the composition includes an isolated nucleic acid sequence having at least about 45% sequence identity to a sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar or identical secondary structure to SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:14, SEQ ID NO:15, SEQ ID NO:16, SEQ ID NO:17, SEQ ID NO:18, SEQ ID NO:19, or SEQ ID NO:20. In certain embodiments, the hepatitis C virus has a genotype selected from the group consisting of 1a, 1b, 2a, 2b, 2c, or 3a.

Methods for treating a patient having a hepatitis C virus are provided, wherein the methods include administering to the patient a composition comprising a therapeutically effective amount of an isolated nucleic acid sequence that has a similar or identical secondary structure to a native NS5B, X, BA, or EC region of a hepatitis C viral RNA. In certain embodiments, the nucleic acid sequence does not have significant sequence identity to the native region. In one embodiment, the nucleic acid sequence has a similar or identical predicted secondary structure to a native NS5B region, but does not have significant sequence identity to the NS5B region. In other embodiments, the methods for inhibiting replication of a hepatitis C virus include adding a composition to the virus, wherein the composition includes an isolated nucleic acid sequence having at least about 45% sequence identity to a sequence selected from the group consisting of SEQ ID NO:1, SEQ ID NO:2, SEQ ID NO:3, SEQ ID NO:4, SEQ ID NO:5, SEQ ID NO:8, SEQ ID NO:9, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar or identical secondary structure to SEQ ID NO:1, SEQ ID NO:2, SEQ ID NO:3, SEQ ID NO:4, SEQ ID NO:5, SEQ ID NO:8, SEQ ID NO:9, SEQ ID NO:14, SEQ ID NO:15, SEQ ID NO:16, SEQ ID NO:17, SEQ ID NO:18, SEQ ID NO:19, or SEQ ID NO:20. In certain embodiments, the patient has hepatitis C virus 1b or 2a. In other embodiments, the patient has hepatitis C virus particles of two or more genotypes or subtypes. The present methods of treatment may further include administering to the patient an additional agent for the treatment of hepatitis C infection. In certain embodiments, the additional agent is a pegylated interferon or ribavirin. The present methods of treatment may include the use of compositions that contain at least two different isolated nucleic acid sequences, wherein each isolated nucleic acid sequence has a similar or identical secondary structure to a native NS5B, X, BA, or EC region of a hepatitis C viral RNA.

Also provided are methods for identifying a nucleic acid sequence that inhibits viral replication, including: selecting a target sequence on a native viral nucleic acid, wherein the presence of additional copies of such target sequence affects viral replication; using a computer program to predict the secondary or tertiary structure of the target sequence; and identifying a nucleic acid sequence that has a similar or identical predicted secondary or tertiary structure to the target sequence. In certain embodiments, the native viral nucleic acid is a hepatitis C viral (HCV) RNA. In certain embodiments, the target sequence is a native NS5B, X, BA, or EC region of the HCV RNA. The computer program may predict secondary or tertiary structure. In some embodiments, the identified nucleic acid sequence does not have significant sequence similarity to the target sequence.

### BRIEF DESCRIPTION OF THE DRAWINGS

FIG. 1 shows schematic diagrams of the predicted folding of RNA sequences designed as analogs of several regions of the HCV genotype 1b (HCV 1b) viral genome. The diagrams show both the sequence and the predicted secondary structure of the region according to the mfold ver. 3.2 program (Washington University, St. Louis, Mo.). The analogs were predicted to adopt stem-loop structures identical to the corresponding replication sequences in full-length viral RNA. Panel A is a schematic diagram of the NS5B region of the HCV 1b genome (5B-74; SEQ ID NO:1), i.e., the positive strand of a region of the NS5B polymerase. This sequence shares 74% sequence identity with the NS5B region of the HCV genotype 2a genome (SEQ ID NO:3). Panel B is a schematic diagram of the BA region of the HCV 1b genome (SEQ ID NO:8), i.e., 3′ terminus of the negative strand. Panel C is a schematic diagram of the EC region of the HCV 1b genome (SEQ ID NO:6), i.e., the 3′ terminus of the negative strand. Panel D is a schematic diagram of the X region of the HCV 1b genome (SEQ ID NO:4), i.e., the positive strand of the X region.

FIG. 2 is a schematic diagram of an RNA structural analog (5B-46) of the HCV 1b viral genome. The wild type sequence of the NS5B region of HCV 1b is shown in the stem loop structure (SEQ ID NO:1). The specific nucleotide substitutions that were introduced into the 5B-46 molecule (SEQ ID NO:2) are shown in bold outside the stem loop structure. The original base pairs in the stem regions were replaced with different, but complementary bases in the 5B-46 molecule.

FIG. 3 shows a graph of real-time PCR results, demonstrating the effect of different molecules on the replication of HCV, as measured by real-time RT-PCR HCV 1b mRNA levels compared to the mRNA levels in an untreated control. An unrelated hepatitis B (HBV) sequence was used as a negative control. The columns and bars represent the means and standard deviations, respectively, of three independent triplicate transfections. The NS5B molecule significantly and very substantially (more than 90%) inhibited replication while the X molecule and the BA molecule were moderately effective at 50% and 60% inhibition, respectively. The EC molecule actually resulted in increased HCV RNA levels, but this increase was not statistically significant.

FIG. 4 shows a graph of real-time RT-PCR results, showing the effect of 5B molecules with different levels of sequence identity to native HCV 2a sequence on the replication of HCV 2a virus infection model compared to the levels in an unrelated HBV treated control. The 5B-74 molecule (SEQ ID NO:1) contains 74% sequence identity to the same region of the HCV 2a genome. Similarly, the 5B-46 (SEQ ID NO:2) and 5B-100 (SEQ ID NO:3) molecules share 46 and 100% sequence identity, respectively, with the same region of the HCV 2a genome. Assays were performed in triplicate, and results are expressed as means plus standard deviations of HCV replication as percents of unrelated HBV control. The 5B-74 analog decreased HCV replication by 91%. Surprisingly, in spite of a sequence identity of only 46%, the novel analog 5B-46, decreased HCV replication in JFH-1 infected cells by 90%, not significantly different from the analog 5B-74 with 74% identity, or analog 5B-100 with complete identity to HCV genotype 2a which decreased replication by 87%. All three 5B molecules significantly and substantially inhibited the replication of the virus, suggesting that the structure rather than the sequence of the molecules was important.

FIG. 5 is a Western blot, confirming the results of the real-time RT-PCR experiments. Neither the expression of the housekeeping protein tubulin, (lane 1, panel B) nor the NS3 protease (lane 1, panel A) was affected by the unrelated HBV control analog. However, the 5B-74 analog decreased NS3 protease levels by more than 90% (lane 3, panel A), while tubulin levels remained unchanged (lane 3, panel B).

FIG. 6 is a schematic diagram showing the sequence of four different antisense RNA molecules in the NS5B region (5B-C1 (SEQ ID NO:10); 5B-C2 (SEQ ID NO:11); 5B-C3 (SEQ ID NO:12); and 5B-C4 (SEQ ID NO:13)). Analysis by the mfold ver 3.2 program indicated that these sequences failed to generate any stem-loop structures resembling the corresponding regions in the native 5B-74 (data not shown).

FIG. 7 is a graph of real-time RT-PCR results, showing the effect of 5B antisense molecules on the replication of HCV 2a virus as measured by HCV mRNA levels compared to the levels in an unrelated HBV treated control. Assays were performed in triplicate, and results are expressed as means plus standard deviations of HCV replication as percents of unrelated HBV control. None of the antisense molecules had a significant impact on the viral replication.

FIG. 8 shows schematic diagrams of the predicted folding of RNA sequences designed as analogs of the NS5B region of several different the HCV genotypes or subtypes. The diagrams show both the sequence and the predicted secondary structure of the region according to the mfold ver. 3.2 program (Washington University, St. Louis, Mo.). Panel A is a schematic diagram of the NS5B region of the HCV 2a genome (SEQ ID NO:3), i.e., the positive strand of a region of the NS5B polymerase. Panel B is a schematic diagram of the NS5B region of the HCV 2b genome (SEQ ID NO:15). Panel C is a schematic diagram of the NS5B region of the HCV 2c genome (SEQ ID NO:16). Panel D is a schematic diagram of the NS5B region of the HCV 3a genome (SEQ ID NO:17). Panel E is a schematic diagram of the NS5B region of the HCV 4 genome (SEQ ID NO:18). Panel F is a schematic diagram of the NS5B region of the HCV 5 genome (SEQ ID NO:19). Panel G is a schematic diagram of the NS5B region of the HCV 6 genome (SEQ ID NO:20).

### DETAILED DESCRIPTION

The present compositions and methods may be understood more readily by reference to the following detailed description of the preferred embodiments and the Examples included herein. However, before the present compositions and methods are disclosed and described, it is to be understood that the disclosed compositions and methods is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting. It is further to be understood that unless specifically defined herein, the terminology used herein is to be given its traditional meaning as known in the relative art.

Described herein is the discovery of a class of polynucleotides that can inhibit viral replication and that are designed to mimic the secondary and tertiary structure of natural viral nucleic acids, without having substantial nucleic acid sequence identity. In particular, compositions and methods for the inhibition of hepatitis C virus (HCV) replication are provided. Specific sequences are offered that dramatically inhibit HCV replication without having substantial HCV sequence identity. This is a novel approach to antiviral therapy, and may be applicable not only to HCV polymerase, but to other HCV targets, and other viruses as well.

In some embodiments, the compositions may comprise a nucleic acid sequence that is identical to a native region of the hepatitis C virus (HCV) genome. In other embodiments, the compositions may comprise a nucleic acid sequence that has at least about 45% to about 95% sequence identity to the native HCV sequence and that has a similar predicted structure to the native HCV sequence. In certain embodiments, a native NS5B sequence may be selected from the group consisting of HCV genotype 1b (SEQ ID NO:1), 2a (SEQ ID NO:3), 1a (SEQ ID NO:14), 2b (SEQ ID NO:15), 2c (SEQ ID NO:16), 3a (SEQ ID NO:17), 4 (SEQ ID NO:18), 5 (SEQ ID NO:19), and 6 (SEQ ID NO:20), As used herein, such identical and similar isolated nucleic acid sequences may be referred to as being an “analog,” a “structural analog,” or a “mimic,” for example, and they refer to nucleic acid sequences that have a similar predicted secondary structure as the native viral sequence. In some embodiments, the predicted secondary structure consists of one or more “stem-loop” structures in which nucleotides form a bond with other nucleotides in the stem portion of the structure. In certain embodiments, the compositions may comprise a nucleic acid sequence that is about 95 bases in length, about 98 bases in length, about 104 bases in length, or about 116 bases in length, and that has a similar predicted structure to the native HCV NS5B, X, BA, or EC sequence, respectively. In such embodiments, the nucleic acid sequence may or may not have sequence similarity with the native HCV sequence. A computer program such as the mfold ver. 3.2 (Washington University, St. Louis, Mo.) may be used to determine whether a sequence is predicted to have a similar predicted structure to the native HCV sequence. In certain embodiments, the isolated nucleic acid sequence differs from the native HCV NS5B, X, BA, or EC sequence in a sequence which forms a stem of a stem-loop secondary structure. For example, the isolated sequence may differ by at least one nucleotide in the sequence which forms the stem of the stem-loop structure.

As used herein, the term “nucleic acid” and “polynucleotide” refer to RNA or DNA that is linear or branched, single or double stranded, or a hybrid thereof. The term also encompasses RNA/DNA hybrids. In a preferred embodiment, the nucleic acid or polynucleotide is a single stranded RNA molecule. These terms include coding regions and also encompass untranslated sequences located at both the 3′ and 5′ ends of the coding region of a gene. Less common bases, such as inosine, 5-methylcytosine, 6-methyladenine, hypoxanthine, and others can also be used. Other modifications, such as modification to the phosphodiester backbone, or the 2′-hydroxy in the ribose sugar group of the RNA can also be made. The polynucleotides can consist entirely of ribonucleotides, or can contain mixed ribonucleotides and deoxyribonucleotides. The polynucleotides may be produced by any means, including genomic preparations, cDNA preparations, in vitro synthesis, RT-PCR, in vitro or in vivo transcription, and viral transduction.

The disclosed nucleic acid molecules, or a portion thereof, can be isolated using standard molecular biology techniques and the sequence information provided herein. For example, a nucleic acid molecule can be amplified using genomic RNA as a template and appropriate oligonucleotide primers according to standard PCR amplification techniques. Furthermore, polynucleotides corresponding to a particular HCV nucleic acid sequence can be prepared by standard synthetic techniques, e.g., using an automated DNA synthesizer. As also used herein, the terms “peptide,” “polypeptide,” and “protein” refer to a chain of at least four amino acids joined by peptide bonds. The chain may be linear, branched, circular, or combinations thereof.

The percent sequence identity between the two sequences is a function of the number of identical positions shared by the sequences (i.e., percent sequence identity=numbers of identical positions/total numbers of positions×100). In some embodiments, the isolated nucleic acid sequence is 100% identical to the native HCV sequence. In other embodiments, the isolated nucleic acid sequences are between about 45% and about 95% identical to an entire amino acid sequence selected from the group consisting of SEQ ID NO:1, SEQ ID NO:2, SEQ ID NO:4, SEQ ID NO:8, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar secondary structure to a sequence that is 100% identical to SEQ ID NO:1, SEQ ID NO:2, SEQ ID NO:4, SEQ ID NO:5, SEQ ID NO:8, SEQ ID NO:14, SEQ ID NO:15, SEQ ID NO:16, SEQ ID NO:17, SEQ ID NO:18, SEQ ID NO:19, or SEQ ID NO:20. In another embodiment, the isolated nucleic acid sequence is between about 45% and 73% or between 75% and about 95% sequence identity to a sequence selected from the group consisting of SEQ ID NO:3, SEQ ID NO:5, and SEQ ID NO:9, wherein the isolated nucleic acid sequence has a similar secondary structure to a sequence that is 100% identical to SEQ ID NO:3, SEQ ID NO:5, or SEQ ID NO:9. In certain embodiments, the sequence differences between the isolated nucleic acid sequence and the native HCV sequence are located in a portion of the sequence that forms a stem region of a stem-loop structure. For example, the isolated nucleic acid sequence may differ by at least one nucleotide in the portion of the sequence that forms the stem region of the stem-loop structure. In other embodiments, the isolated nucleic acid sequence is about 95 bases in length, about 98 bases in length, about 104 bases in length, or about 116 bases in length, and has a similar predicted structure to the native HCV NS5B, X, BA, or EC sequence, respectively, without having significant sequence similarity with the native HCV sequence. As used herein, an isolated nucleic acid sequence that does not have “significant sequence similarity” with a native HCV sequence has less than 50% sequence identity with the native HCV sequence. In certain embodiments, the isolated nucleic acid sequence has less than 40%, less than 30%, or less than 20% sequence identity with the native HCV sequence. In one embodiment, the isolated nucleic acid sequence has 46% sequence identity with the native HCV NS5B sequence.

For the purposes of designing structural analogs of HCV RNA sequences, the mfold ver 3.2 program (Washington University, St. Louis, Mo.) may be used to identify structural analogs with base substitutions that are predicted to have secondary structures identical or similar to the secondary structures of native molecules. The mfold ver 3.2 program (Washington University, St. Louis, Mo.) also may be used to identify structural analogs with a thermodynamically predicted free energy identical to or within 0.1 kCal of the free energy of the native structure.

Methods for inhibiting replication of a hepatitis C virus are provided which comprise adding to the virus one of the presently disclosed compositions comprising a nucleic acid molecule that has a similar secondary structure to the native HCV sequence. The HCV may be a genotype selected from the group consisting of 1, 2, 3, 4, 5, or 6. In some embodiments, the HCV is an HCV genotype 1 or genotype 2. The HCV may be of a subtype selected from the group consisting of 1a, 1b, 2a, 2b, 2c, or 3a. In certain other embodiments, the HCV is subtype 1b or subtype 2a.

The presently disclosed compositions comprising a nucleic acid molecule that has a similar secondary structure to the native HCV sequence may be used for the manufacture of a medicament for the treatment of patients with an HCV infection. The compositions may further comprise a pharmaceutically acceptable carrier. The phrases “pharmaceutically or pharmacologically acceptable” refer to molecular entities and compositions that do not produce an adverse, allergic, or other untoward reaction when administered to an animal, or a human, as appropriate. Veterinary uses are equally included, and “pharmaceutically acceptable” formulations include formulations for both clinical and/or veterinary use. As used herein, “pharmaceutically acceptable carrier” includes any and all solvents, dispersion media, coatings, antibacterial, and antifungal agents, isotonic and absorption delaying agents, and the like. The use of such media and agents for pharmaceutically active substances is well known in the art. Except insofar as any conventional media or agent is incompatible with the active ingredient, its use in the therapeutic compositions is contemplated. For human administration, preparations should meet sterility, pyrogenicity, and general safety and purity standards as required by FDA Office of Biologics standards. Supplementary active ingredients can also be incorporated into the compositions.

Also provided are methods for the treatment of a patient having an HCV infection comprising administering one or more of the compositions described herein, and uses of the compositions described herein in the manufacture of a medicament for the inhibition of HCV replication. In certain embodiments, the patient also may be treated with additional HCV inhibiting agents. For example, the one or more additional agents may include pegylated interferon, small molecules, and/or ribavirin. As used herein with respect to these methods, the term “administering” refers to various means of introducing a composition into a cell or into a patient. These means are well known in the art and may include, for example, injection; tablets, pills, capsules, or other solids for oral administration; nasal solutions or sprays; aerosols or inhalants; topical formulations; liposomal forms; and the like. As used herein, the term “effective amount” refers to an amount that will result in the desired result and may readily be determined by one of ordinary skill in the art. For example, in certain embodiments, an effective amount is an amount that would decrease viral replication by at least 10%.

The present compositions (e.g., viral replication-inhibiting) may be formulated for various means of administration. As used herein, the term “route” of administration is intended to include, but is not limited to subcutaneous injection, intravenous injection, intraocular injection, intradermal injection, intramuscular injection, intraperitoneal injection, intratracheal administration, epidural administration, inhalation, intranasal administration, oral administration, sublingual administration, buccal administration, rectal administration, vaginal administration, and topical administration.

If the composition is to be administered via injection, the preparation of an aqueous composition that contains an HCV nucleic acid as an active ingredient will be known to those of skill in the art. Typically, such compositions can be prepared as injectables, either as liquid solutions or suspensions; solid forms suitable for using to prepare solutions or suspensions upon the addition of a liquid prior to injection can also be prepared; and the preparations can also be emulsified. The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases, the form should be sterile and fluid to the extent that syringability exists. It should be stable under the conditions of manufacture and storage and should be preserved against the contaminating action of microorganisms, such as bacteria and fungi.

The disclosed compositions can be formulated into a sterile aqueous composition in a neutral or salt form. Solutions as free base or pharmacologically acceptable salts can be prepared in water suitably mixed with a surfactant, such as hydroxypropylcellulose. Pharmaceutically acceptable salts, include the acid addition salts (formed with the free amino groups of the protein), and those that are formed with inorganic acids such as, for example, hydrochloric or phosphoric acids, or such organic acids as acetic, trifluoroacetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine, and the like.

Suitable carriers include solvents and dispersion media containing, for example, water, ethanol, polyol (for example, glycerol, propylene glycol, and liquid polyethylene glycol, and the like), suitable mixtures thereof, and vegetable oils. In many cases, it will be preferable to include isotonic agents, for example, sugars, or sodium chloride. The proper fluidity can be maintained, for example, by the use of a coating, such as lecithin, by the maintenance of the required particle size in the case of dispersion and/or by the use of surfactants.

Under ordinary conditions of storage and use, all such preparations should contain a preservative to prevent the growth of microorganisms. The prevention of the action of microorganisms can be brought about by various antibacterial and antifungal agents, for example, parabens, chlorobutanol, phenol, sorbic acid, thimerosal, and the like. Prolonged absorption of the injectable compositions can be brought about by the use in the compositions of agents delaying absorption, for example, aluminum monostearate, and gelatin.

Prior to or upon formulation, the present compositions should be extensively dialyzed to remove undesired small molecular weight molecules, and/or lyophilized for more ready formulation into a desired vehicle, where appropriate. Sterile injectable solutions are prepared by incorporating the active agents in the required amount in the appropriate solvent with various of the other ingredients enumerated above, as desired, followed by filter sterilization. Generally, dispersions are prepared by incorporating the various sterilized active ingredients into a sterile vehicle that contains the basic dispersion medium and the required other ingredients from those enumerated above.

In the case of sterile powders for the preparation of sterile injectable solutions, the preferred methods of preparation are vacuum-drying and freeze-drying techniques that yield a powder of the active ingredient, plus any additional desired ingredient from a previously sterile-filtered solution thereof.

Suitable pharmaceutical compositions in accordance with the disclosure will generally include an amount of the active ingredient admixed with an acceptable pharmaceutical diluent or excipient, such as a sterile aqueous solution, to give a range of final concentrations, depending on the intended use. The techniques of preparation are generally well known in the art as exemplified by Remington's Pharmaceutical Sciences, 16th Ed. Mack Publishing Company, 1980, incorporated herein by reference. It should be appreciated that for human administration, preparations should meet sterility, pyrogenicity, and general safety and purity standards as required by FDA Office of Biological Standards.

Also provided are methods for identifying nucleic acid sequences that inhibit viral replication. These methods comprise selecting a target sequence on a native viral nucleic acid, wherein the presence of additional copies of such target sequence affects viral replication; using a computer program to predict the secondary or tertiary structure of the target sequence; and identifying a nucleic acid sequence that has a similar or identical predicted secondary or tertiary structure to the target sequence. The computer program may predict secondary structure. In one embodiment, the mfold ver. 3.2 program is used. Such methods may be used to identify nucleic acid sequences of any virus. In a preferred embodiment, the virus is hepatitis C virus. The isolated nucleic acids identified using these methods have a similar or identical predicted secondary or tertiary structure, but may or may not have significant sequence similarity to the target sequence.

Throughout this application, various publications, patents, and patent applications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments and that numerous changes may be made therein without departing from the scope of the disclosed compositions and methods. The compositions and methods are further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present compositions and methods and/or the scope of the appended claims.

### EXAMPLES

### Example 1

### Identification and Production of Target Nucleic Acid Structural Analogs

The crucial interaction in HCV replication is the binding of nucleic acids by the polymerase. Because physical interaction with the RNA is required, it seemed possible that analogs could be created that would sufficiently resemble the natural genomic structure as to compete with the HCV genome, resulting in inhibition of replication. Therefore, we designed several polynucleotides to mimic the secondary structure of native viral nucleic acids, without requiring substantial sequence identity to the native nucleic acid sequences.

HCV RNA has a number of cis-acting replication elements (CREs) whose function could potentially be inhibited by structural RNA analogs. For example, structural analogs based on the HCV internal ribosome entry site (IRES) were recently shown to inhibit HCV translation in vitro and in replicon cell culture (Ray and Das, 2004, Nucleic Acids Research, 32:1678-87). In addition to the IRES, HCV RNA bears CREs in the positive-strand NS5B coding region and X region, as well as in the negative strand 3′-terminal region. The function of each CRE is assumed to depend, at least in part, on the reported ability of these structures to bind a variety of host factors and viral non-structural proteins.

The target regions analyzed included the following:

NS5B—As used herein, the terms “NS5B” or “5B” refer to the polymerase gene region in the (+) strand of the HCV genome. It has been shown that HCV polymerase binds to the 5′ of the polymerase gene (Lee et al., 2004, J. Virology, 78:10865-77). The “stem loop 3.2” has been shown to be critical for replication (Friebe et al., 2005, J. Virology, 79: 380-92).

X—promoter for (+) strand synthesis

Negative strand NS3B 3′-terminus—including the BA and EC regions.

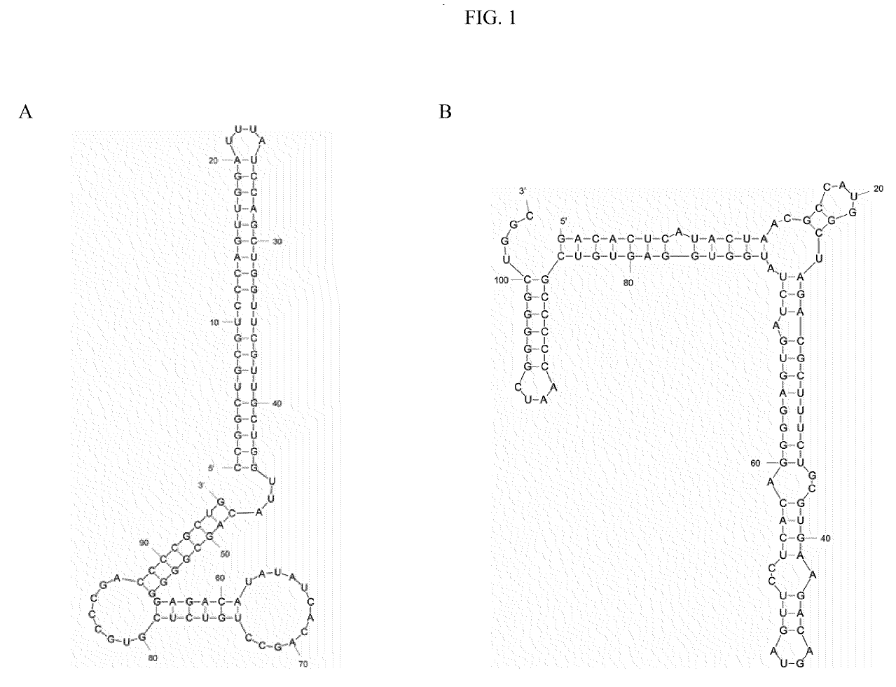

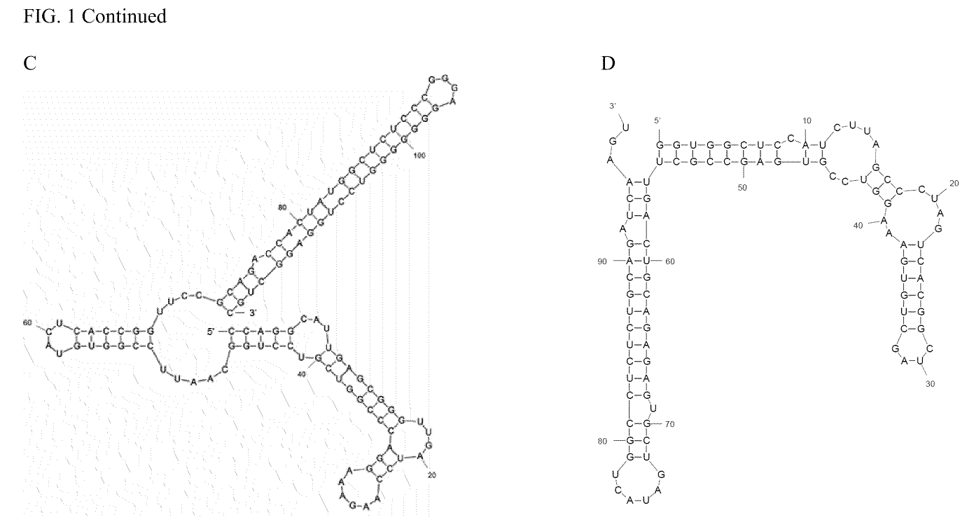

Because typical HCV does not infect cultured cells, a replicon system, HCV 1b subgenomic replicon, BB7, consisting of genes required for HCV RNA replication was used. This system is convenient and reproducible, but does not produce viral particles. Structural RNA analogs were constructed based on RNA sequences predicted to adopt stem-loop structures identical to the corresponding cis-acting replication element in full-length viral RNA, using mfold ver 3.2 (Washington University, St. Louis, Mo.)(FIG. 1A-1D).

Because HCV RNA replication involves generation of a negative strand template, structural analogs were designed for both negative and positive strands to determine differences in efficacy. Table 1 shows the target regions of the BB7 replicon RNA (genotype 1b) towards which analogs were directed. The 5B-74 and X sequences are analogs of regions in the positive strand of the NS5B polymerase gene. EC and BA sequences are analogs of the negative strand of the NS3 protease.

TABLE 1

| RNA structural | Source BB7 | | |
|---|---|---|---|
| analog name | sequence (nt) | Replicon strand | Stem-loop domains |
| 5B-74 | 7600-7694 | (+) strand | NS5B coding region |
| | | | SL3.1, 3.2 |
| X | 7891-7989 | (+) strand | X region SL3, 2, 1 |
| EC | 107-222 | (−) strand | NS3B 3′-terminus SL-EI, |
| | | | DI, CI |
| BA | 1-104 | (−) strand | NS3B 3′-terminus SL-BI, |
| | | | AI |

For replicon studies, analog RNAs were generated in situ by transfection with a plasmid encoding a structural analog cloned into a T7 transcription vector, pENT7, by BamH I/Hind III digestion and ligation Inhibitory activity of each analog was assessed under multiple dosing conditions as discussed further below. Analog inhibitory activity in the cell-free assay was evaluated by transient transfection in the replicon cell culture model by real-time RT-PCR as discussed further below. The HCV RNA structural analog sequences were predicted to adopt stem-loop structures identical to the corresponding cis-acting replication element in full-length viral RNA, as determined by mfold ver. 3.2 web server (Washington University, St. Louis, Mo.)(FIG. 1).

For infection studies, structural analogs of the 5B (polymerase) region that were identical in sequence to the native sequences were constructed based on the HCV 1b subgenomic replicon, BB7. The sequences were predicted using mfold ver 3.2 (Washington University, St. Louis, Mo.) to adopt stem-loop structures identical to the corresponding cis-acting replication element in full-length viral RNA.

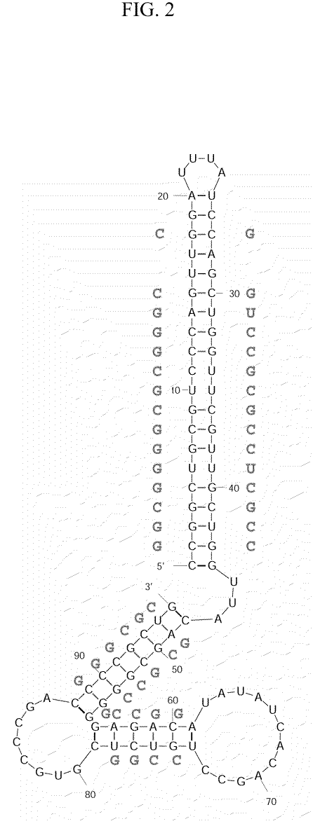

The intent of the design of the molecules was to make a single short single-stranded version of the native structure to compete for protein interactions. However, because the NS5B analog is single (+) stranded, it is possible that observed inhibitory effects could have been due to antisense effects against the negative strand, and not due to conformation. To determine whether this was the case, a new analog was prepared in which bases involved in the stem loops were changed such that the sequence of the stems were 74% different, and overall identity was decreased to 46%, relative to JFH-1 (genotype 2) as shown in FIG. 2. To construct 5B-46, base pair-exchanges were made at positions 2-15, 18, 49-54, 57-60, 75-78, 89-94 in 5B-74. Regions in the stems or loops in which changes were predicted by mfold ver. 3.2 (Washington University, St. Louis, Mo.) to alter secondary structure were left intact.

DNA fragments containing each sequence flanked by BamH I and Hind III sites were generated by PCR amplification of sequences from pHCV rep1bBB7. As a negative control, the 60 nucleotide hepatitis B virus encapsidation signal sequence was amplified by PCR from plasmid adwR9 (a gift from T. Jake Liang, NIH). This region includes HBV sequences flanking the minimal 60 nucleotide element, so that the total insert length approximates that of the analog HCV molecules. The insert was cloned into a T7 transcription vector, pENT7 by BamH I/Hind III digestion and ligation. pENT7 is a derivative of pENTR4 (Invitrogen, Carlsbad, Calif.), in which a T7 expression cassette (BamH I and Hind III sites downstream of the consensus T7 RNA polymerase promoter) has been inserted in the multiple cloning site. For initial clone verification, the restriction enzymes BamH I and Hind III were used. The reaction products were visualized by 1% agarose gel electrophoresis.

For expression of HCV RNA structural analogs as polymerase II transcripts in mammalian cells, each insert was subcloned into pSilencer 4.1-CMV puro (Ambion, Austin, Tex.) by BamH I/Hind III digestion and ligation. To confirm absence of unwanted mutations, putative clones containing pSilencer 4.1-CMV puro with HCV RNA structural analogs were sequenced with sequencing primers: 5′-AGGCGATTAAGTTGGGTA-3′ (SEQ ID NO:21), and 5′-CGGTAGGCGTGTACGGTG-3′ (SEQ ID NO:22).

As a positive control for HCV genotype 2a inhibition, an RNA was constructed to be identical to the 5B region of the HCV genotype 2a virus. The fragment was predicted using mfold ver 3.2 (Washington University, St. Louis, Mo.), to adopt stem-loop structures identical to the corresponding cis-acting replication element in full-length viral RNA.

### Example 2

### Comparison of Inhibitory Effects of Native Sequences and Structural Analogs on HCV Replication

The HCV genotype 1b subgenomic replicon BB7 described above, non-infectious system was used to analyze the effect of the HCV 1b sequences described above on replication inhibition of HCV 1b. An HCV genotype 2a infectious viral system, JFH-1, was used to determine the cross-genotype activity of the sequences, and their inhibition of virus production.

### Cells

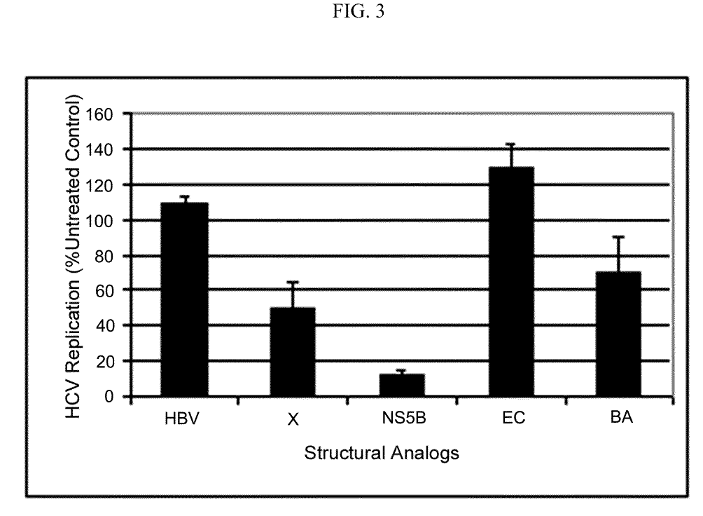

Huh7.5 cells (a generous gift from Dr. Charles M. Rice, Rockefeller University, New York, N.Y.), a human hepatoma cell line which supports viral replication to a high level was maintained in Dulbecco's Modified Eagle Medium (DMEM) with 10% fetal bovine serum (FBS) and antibiotic/antimycotic solution. Cells were passaged every 3-4 days to maintain 75% confluent conditions. The results of the transfection of plasmids encoding RNA analogs into BB7 replicon cells followed by quantitation of HCV RNA by real-time RT-PCR with SYBR GREEN according to the protocol supplied by the manufacturer (Roche Applied Science, Indianapolis, Ind.) using HCV genotype 1b specific primers: forward primer: 5′-CTG TCT TCA CGC AGA AAG CG-3′ (SEQ ID NO:27) and reverse primer: 5′-CAC TCG CAA GCA CCC TAT CA-3′ (SEQ ID NO:28). The results are shown in FIG. 3. The NS5B analog inhibited HCV replication by more than 90%. The other analogs, to X and BA regions, decreased replication by 50% and 60%, respectively. The EC analog actually increased replication, but this increase was not statistically significant.

### Assays of Infection by HCV JFH-1

Because the NS5B structural analog appeared to be the most potent, further experiments were focused on this fragment. To determine whether the analog might be effective against a different HCV genotype and in an infection model, Huh-7.5 cells carrying persistent replicating JFH-1 RNA were seeded 6 days before transfection. For plasmid transfection, 25 μg of each structural analog was transfected into Huh-7.5 cells. The HCV 1b NS5B analog had 74% identity with genotype 2a sequence. Efficiency of RNA replication in the transfected cells was determined by real-time reverse transcription polymerase chain reaction (real-time RT-PCR) using HCV specific primers. Human lactate dehydrogenase A (LDHA) mRNA level in each sample was simultaneously quantified to normalize the values of HCV RNA.

A cDNA to hepatitis C genotype 2a virus with the JFH-1 strain (a generous gift from Dr. Takaji Wakita, National Institute of Infectious Diseases, Tokyo, Japan) was used for transfection of the full-length JFH-1 genome into Huh7.5 cells according to previously published protocol (Wakita et al., 2005, Nat. Med., 11:791-96). Efficiency of RNA replication in the transfected cells was determined by real-time reverse transcription polymerase chain reaction (real-time PCR) using HCV specific primers. The primer sequences were for forward primer 5′-TAG GAG GGC CCA TGT TCA AC-3′ (SEQ ID NO:23) and reverse primer 5′-CCC CTG GCT TTC TGA GAT GAC-3′ (SEQ ID NO:24). The PCR conditions were: 2 min. at 50° C., 10 min. at 95° C. and 15 sec. at 95° C. After 40 cycles, final extension was performed at 60° C. for 1 min.

### Transfection of Plasmids Encoding HCV Structural Analogs

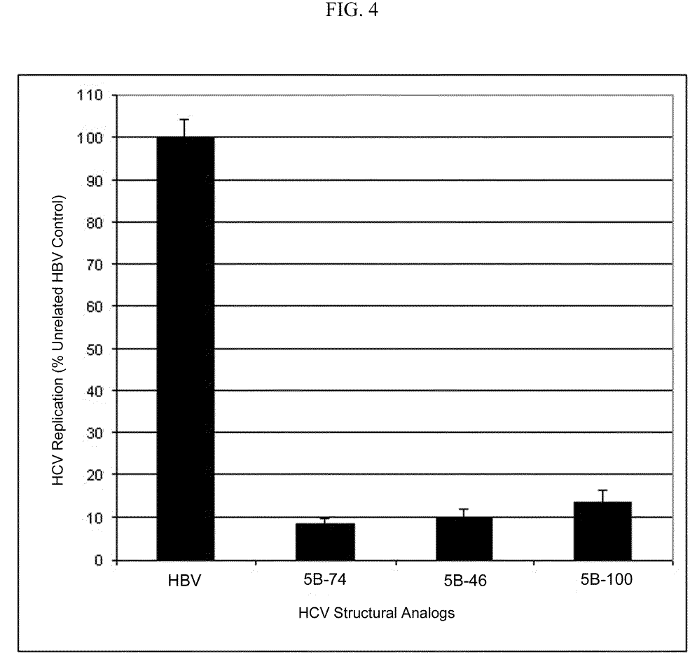

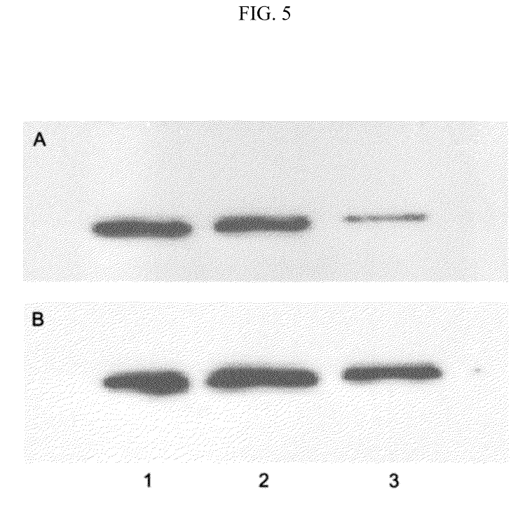

Huh-7.5 cells carrying persistent replicating viral RNA were seeded at a density of 10⁵ cells per well onto 6-well plates 6 days before transfection. For plasmid transfection, 25 μg of each was transfected into Huh-7.5 cells. In brief, six days before transfection, cells were plated in 2 ml of growth medium such that they were 95% confluent at the time of transfection. Plasmid DNA was diluted in 250 μl of Opti-MEM® I Reduced Serum Medium without serum. Lipofectamine™ 2000 was mixed gently before use and diluted to the appropriate amount in 250 μl of Opti-MEM® I Medium. After 5 min. incubation, the diluted DNA was combined with diluted Lipofectamine™ 2000, mixed gently and incubated for 20 min. at 25° C. Complexes, 500 μl, were added to each well containing cells and medium. After 6 hrs of incubation at 37° C. under 5% CO2, 1.5 ml of DMEM and 15% FBS were added to each well. Cells were harvested 48 and 72 hrs after transfection, and HCV RNA levels and NS3 protein in cell lysates were determined by real-time PCR (FIG. 4) and Western blot analyses (FIG. 5), respectively.

### Quantitation of HCV RNA by Real Time RT-PCR

RNAs were isolated from cultured cells with Trizol reagent (Invitrogen, Carlsbad, Calif.), and treated with RNase-free DNase (Promega). One μg of DNase-treated total RNA was reverse transcribed using iScript cDNA Synthesis Kit (BioRad Laboratories, Hercules, Calif.). After incubation at 25° C. for 5 min., at 42° C. for 30 min. and at 85° C. for 5 min., the resulting cDNA was quantified with SYBR GREEN according to the manufacturer's instructions (Roche Applied Science, Indianapolis, Ind.). HCV RNA replication in the transfected cells was determined by real-time PCR using HCV specific primers as described above.

FIG. 4 shows the effects of the structural analogs on JFH-1 viral replication as determined by real time RT-PCR using HCV 2a specific primers. The sequences for the forward primer were: 5′-TAG GAG GGC CCA TGT TCA AC-3′ (SEQ ID NO:23) and reverse primer 5′-CCC CTG GCT TTC TGA GAT GAC-3′ (SEQ ID NO:24). The PCR conditions were: 2 min. at 50° C., 10 min. at 95° C. and 15 sec. at 95° C. After 40 cycles, final extension was performed at 60° C. for 1 min. Assays were performed in triplicate and results expressed as means+S.D. of HCV replication as a percent of unrelated HBV control. As seen previously, the 5B-74 molecule decreased HCV replication to less than 10% of controls. In spite of the decrease in sequence identity to only 46%, the novel analog, 5B-46, decreased HCV replication in JFH-1 infected cells by 91.5%, not significantly different from the analog, 5B-74 with 74% identity (91.2%), or an analog 5B-100 with complete identity to HCV genotype 2a.

Human lactate dehydrogenase A (LDHA) mRNA level in each sample was simultaneously quantified to normalize the values of HCV RNA. The primer sequences were LDHA forward primer 5′-TAA TGA AGG ACT TGG CAG ATG AAC T-3′ (SEQ ID NO:25) and LDHA reverse primer 5′-ACG GCT TTC TCC CTC TTG CT-3′ (SEQ ID NO:26).

FIG. 4 shows the effects of transfection of the NS5B analog 5B-74 on JFH-1 genotype 2a viral infection, as determined by real time PCR. Surprisingly, the 5B-74 analog, which is only 74% identical to the genotype 2a sequence, was as effective in suppression of HCV replication of JFH-1 genotype 2a as in the replicon model for which the analog was 100% identical. The analog against the EC region of the (−) strand was also effective in this system.

### Quantitation of HCV Protein Production by Western Blot Analysis

To determine whether the inhibition of viral replication by the NS5B analog could be confirmed by changes in viral protein synthesis, Western blots to the NS3B protease were performed. Cell debris was removed by centrifugation. Total protein extracts of cell lysates were evaluated by Western blot analysis. Cells were harvested in RIPA buffer (50 mM Tris-HCl, pH 7.4, 1% NP-40, 0.25% Na-deoxycholate, 150 mM NaCl, 1 mM EDTA) supplemented with a protease inhibitor cocktail. Cell debris was removed by centrifugation. Protein concentration was determined with Bio-Rad protein assay. Forty μg of protein was resolved by SDS-PAGE, and transferred to Hybond nitrocellulose membranes. Membranes were sequentially blocked with 5% nonfat milk in PBS, incubated with a 1:1000 dilution of the monoclonal mouse HCV NS3 antibody, washed 3 times with PBS/0.05% Tween 20, incubated with horseradish peroxidase-conjugated goat anti-mouse antibody at 1:20,000 dilution. Bound antibody complexes were detected with SuperSignal chemiluminescent substrate. To ensure comparable loading of the samples, blots were incubated with a 1:1000 dilution of a polyclonal rabbit alpha-tubulin antibody, and horseradish peroxidase conjugated goat anti-rabbit secondary antibody using the same procedures as described above.

FIG. 5 shows the results of this Western blot analysis of tubulin or NS3 protease protein 72 hours after transfection. Lane 1 is the protein level prior to addition of an analog; lane 2 is the protein level in the cells treated with the HBV (unrelated) control; and lane 3 is the protein level in cells treated with the novel NS5B analog. As shown in FIG. 5, neither the expression of the housekeeping gene tubulin, nor the NS3 protease was affected by the unrelated HBV control. However, the novel NS5B analog decreased NS3 protease levels by more than 90%, while tubulin levels remained unchanged.

### Example 3

### Effect of Antisense Molecules on HCV Replication

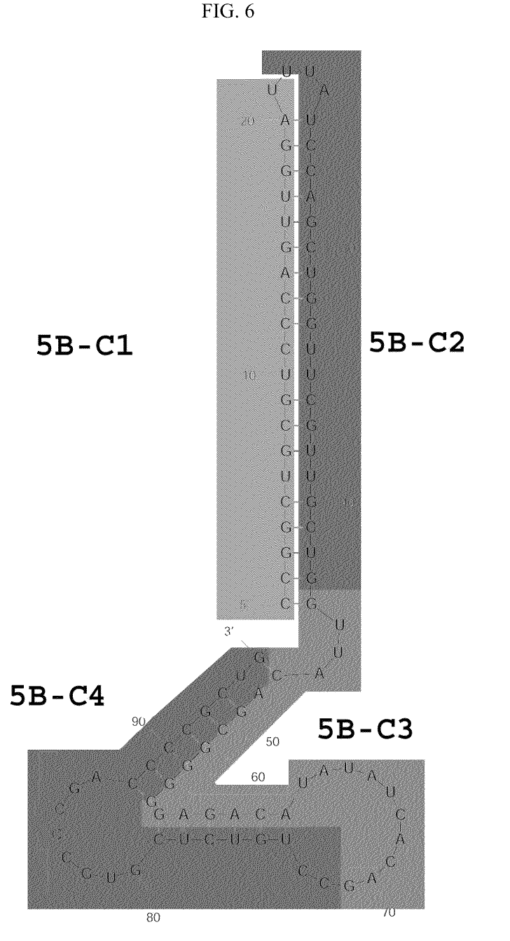

While the stem regions were replaced in the novel analog, the sequences in the loops remained unchanged in the novel analog. Therefore, although unlikely, an antisense effect caused by the loop regions was still possible. The loop regions could not be altered without altering the conformation of the stems. Therefore, to determine whether the loop regions were involved in the observed effects of the novel analog, short fragments spanning the entire analog, containing sequences of the loop regions were prepared, as shown in FIG. 6.

Four complementary RNAs 21-28 nucleotides long, together spanning the entire 5B-74 analog were commercially synthesized. Complementary RNAs sequences were as listed:

| | |
|---|---|
| antisense RNA-5B-C1: | |
| 5′-CCGGCUGCGUCCCAGUUGGAU-3′ | (SEQ ID NO: 10) |
| antisense RNA-5B-C2: | |
| 5′-UUAUCCAGCUGGUUCGUUGCUG-3′ | (SEQ ID NO: 11) |
| antisense RNA-5B-C3: | |
| 5′-GUUACAGCGGGGGAGACAUAUAUCACAG-3′ | (SEQ ID NO: 12) |
| antisense RNA-5B-C4: | |
| 5′-CCUGUCUCGUGCCCGACCCCGCUG-3′. | (SEQ ID NO: 13) |

To determine effects of complementary RNAs on HCV RNA levels in an HCV infection system, these antisense RNAs were transfected according to the transfection protocol described above. Briefly, one day before transfection, cells were plated in the appropriate amount of growth medium without antibiotics such that they were 95% confluent at the time of transfection. For each transfection sample, oligomer-Lipofectamine™ 2000 complexes were prepared by dilution of 50 pmol or 500 pmol RNA oligomer in 250 μl of Opti-MEM® I Reduced Serum Medium without serum. 5 μl of Lipofectamine™ 2000 was diluted in 250 μl Opti-MEM® I Reduced Serum Medium, mixed gently and incubated for 5 minutes at 25° C.

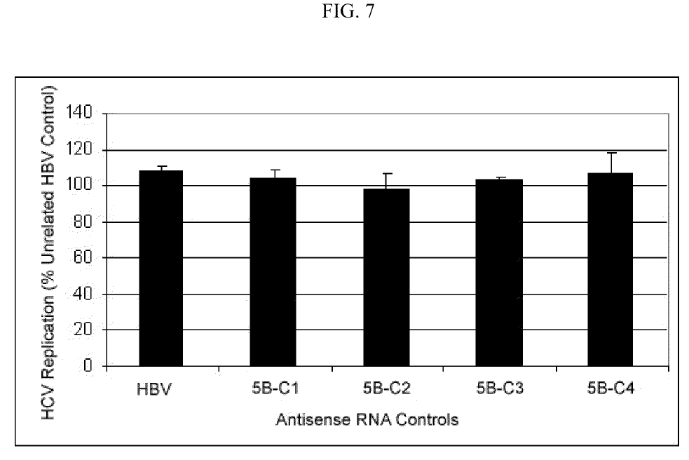

After 5 minutes incubation, the diluted oligomer was combined with diluted Lipofectamine™ 2000, mixed gently, and incubated for 20 minutes at 25° C. to allow complex formation to occur. The oligomer-Lipofectamine™ 2000 complexes were added to each well containing cells and medium, mixed. After 6 hours, 1.5 ml of 15% FBS diluted in DMEM was added to the wells and the cells were incubated at 37° C. in a CO₂ incubator for 48 hours. At this time point, HCV RNA replication in the transfected cells was determined by RT-PCR using HCV specific primers as described above. The effects of these antisense molecules on viral replication as determined by real time PCR is shown in FIG. 7.

FIG. 7 shows the effect of the antisense RNA molecules directed against specific regions of the NS5B region corresponding to the 5B-74 analog, on HCV replication. None of these sequences had any significant effects on HCV RNA levels, as all levels remained at control levels. These results support the conclusion that the 5B-74 and 5B-46 analogs inhibited HCV replication not by sequence complementarity, but by conformational attributes. Implicit in these results is the conclusion that a computer predicted secondary structure can guide the construction of sequences that mimic not only secondary structure for which the program was designed, but also mimic tertiary structure, without which inhibitory interaction with the HCV target would not have been observed. This was also an unexpected result.

### Other Embodiments

The compositions and methods illustratively described herein suitably may be practiced in the absence of any element or elements, limitation, or limitations not specifically disclosed herein. Thus, for example, in each instance herein any of the terms “comprising,” “consisting essentially of,” and “consisting of” may be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the compositions and methods claimed.

It will be readily apparent to one skilled in the art that varying substitutions and modifications may be made to the compositions and methods disclosed herein without departing from the scope and spirit of the disclosure. For example, HCV genotypes and subtypes not listed herein fall within the scope of the present compositions and methods. Thus, such additional embodiments are within the scope of the present compositions and methods, and the following claims.

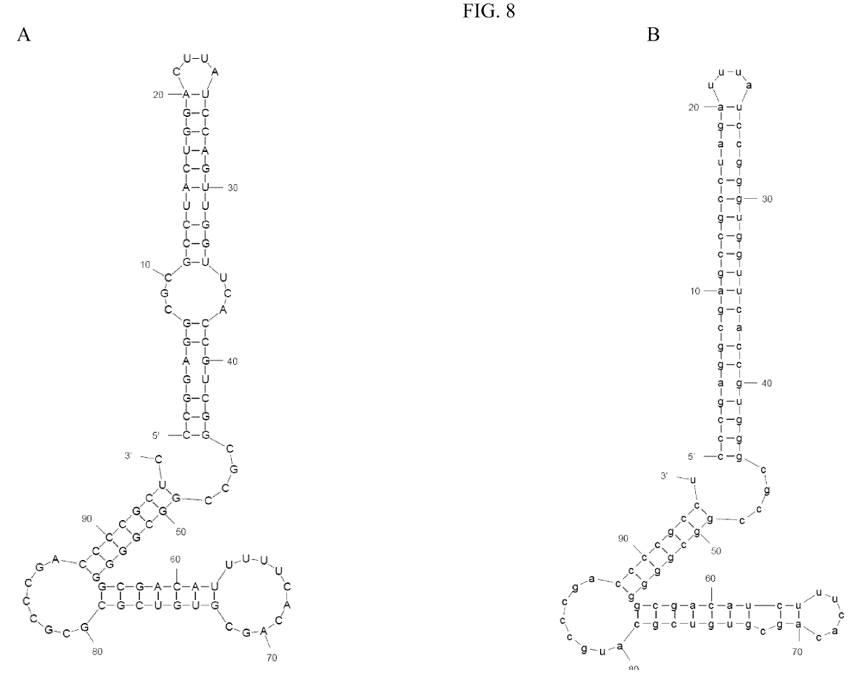

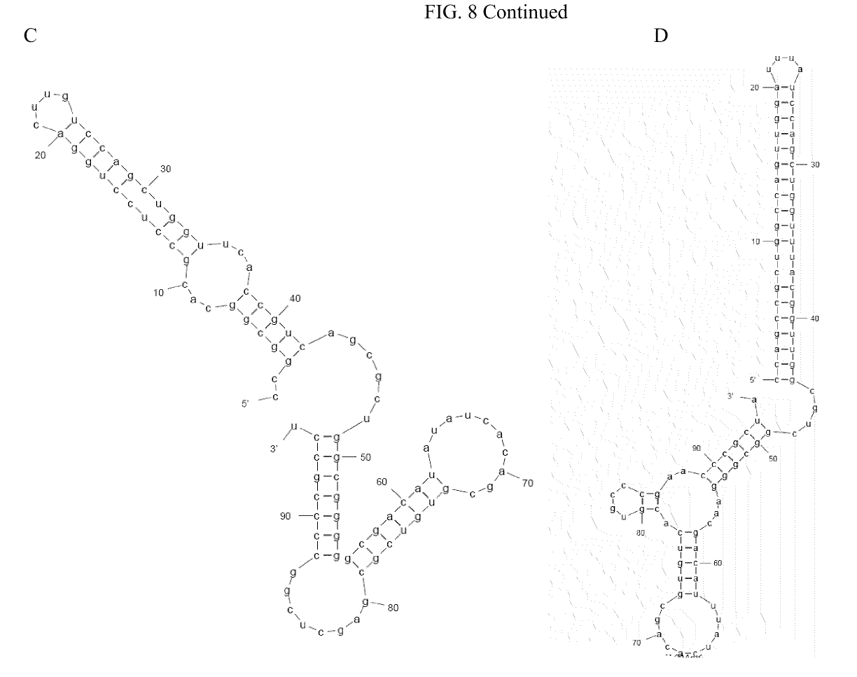

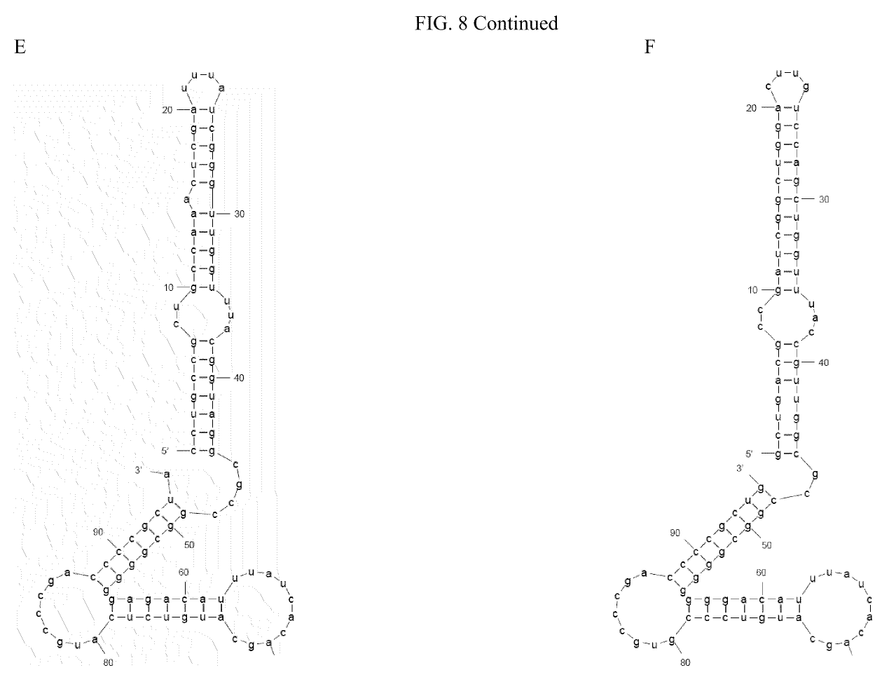

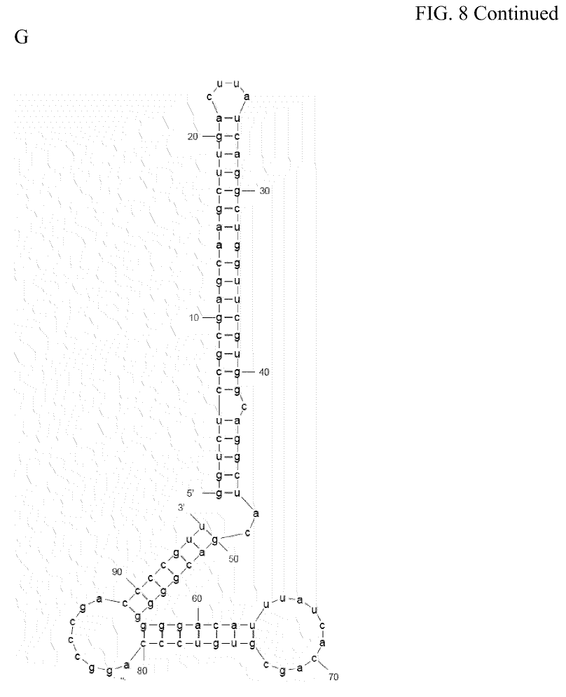

APPENDIX
SEQ ID NO: 1
(FIG. 1A - HCV 1b NS5B region (“5B-74”))
CCGGCUGCGUCCCAGUUGGAUUUAUCCAGCUGGUUCGUUGCUGGUUACAG
CGGGGGAGACAUAUAUCACAGCCUGUCUCGUGCCCGACCCCGCUG
SEQ ID NO: 2
(FIG. 2 - HCV 1b NS5B region (“5B-46”))
CGGCGGGGCGCGGGCUUCGAUUUAUCGAGGUCCGCGCCUCGCCGUUACGC
GCCGGGCCGGAUAUAUCACAGCCUCCGGCGUGCCCGACGGGCGCG
SEQ ID NO: 3
(FIG. 8A - HCV 2a NS5B region (“5B-100”))
CCGGAGGCGCGCCUACUGGACUUAUCCAGUUGGUUCACCGUCGGCGCCGG
CGGGGGCGACAUUUUUCACAGCGUGUCGCGCGCCCGACCCCGCUC
SEQ ID NO: 4
(FIG. 1D - HCV 1b X region)
GGUGGCUCCAUCUUAGCCCUAGUCACGGCUAGCUGUGAAAGGUCCGUGAG
CCGCUUGACUGCAGAGAGUGCUGAUACUGGCCUCUCUGCAGAUCAAGU
SEQ ID NO: 5
(HCV 2a X region)
GGT GGC TCC ATC TTA GCC CTA GTC ACG GCT AGC TGT
GAA AGG TCC GTG AGC CGC ATG ACT GCA GAG AGT GCC
GTA ACT GGT CTC TCT GCA GAT CA
SEQ ID NO: 6
(FIG 1C - HCV 1b EC region)
CCAGGCAUUGAGCGGGUUGAUCCAAGAAAGGACCCGGUCGUCCUGGCAAU
UCCGGUGUACUCACCGGUUCCGCAGACCACUAUGGCUCUCCCGGGAGGGG
GGGUCCUGGAGGCUGC
SEQ ID NO: 7
(HCV 2a EC region)
CCG GGC ATA GAG TGG GTT TAT CCA AGA AAG GAC CCA
GTC TTC CCG GCA ATT CCG GTG TAC TCA CCG GTT CCG
CAG ACC ACT ATG GCT CTC CCG GGA GGG GGG GGC CTG
GAG GCT GT
SEQ ID NO: 8
(FIG 1B - HCV 1b BA region)
GACACUCAUACUAACGCCAUGGCUAGACGCUUUCUGCGUGAAGACAGUAG
UUCCUCACAGGGGAGUGAUCUAUGGUGGAGUGUCGCCCCCAAUCGGGGGC
UGGC
SEQ ID NO: 9
(HCV 2a BA region)
CGA CAC TCA TAC TAA CGC CAT GGC TAG GCG CTT TCT
GCG TGA AGA CAG TAG TTC CTC ACA GGG GAG TGA TTC
ATG GCG GAG TGT CGC CCC TAT TAG GGG CAG GT
SEQ ID NO: 10
antisense RNA - 5B-C1
CCGGCUGCGUCCCAGUUGGAU
SEQ ID NO: 11
antisense RNA - 5B-C2
UUAUCCAGCUGGUUCGUUGCUG
SEQ ID NO: 12
antisense RNA - 5B-C3
GUUACAGCGGGGGAGACAUAUAUCACAG
SEQ ID NO: 13
antisense RNA - 5B-C4
CCUGUCUCGUGCCCGACCCCGCUG
SEQ ID NO: 14
(HCV 1a NS5B region)
GGC CGC TAG CCC AGC TGG ACT TGT CCG GTT GGT TCA
CGG CTG GCT ACA GCG GGG GAG ACA TTT ATC ACA GCG
TGT CTC GTG CCC GGC CCC GCT G
SEQ ID NO: 15
(FIG. 8B - HCV 2b NS5B region)
CCCGAGGCGAGCCGCCUAGAUUUAUCCGGGUGGUUCACCGUGGGCGCCGG
CGGGGGCGACAUCUUUCACAGCGUGUCGCAUGCCCGACCCCGCCU
SEQ ID NO: 16
(FIG. 8C - HCV 2c NS5B region)
CCGGCGGCACGCCUCCUGGACUUGUCCAGCUGGUUCACCGUCAGCGCUGG
CGGGGGCGACAUAUAUCACAGCGUGUCGCGAGCUCGGCCCCGCCU
SEQ ID NO: 17
(FIG. 8D - HCV 3a NS5B region)
CCAGCCGCUGGCCAGUUGGAUUUAUCCAGCUGGUUUACGGUUGGCGUCGG
CGGGAACGACAUUUAUCACAGCGUGUCACGUGCCCGAACCCGCUA
SEQ ID NO: 18
(FIG. 8E - HCV 4 NS5B region)
CCUGCCGCUGCCAAACUCGAUUUAUCGGGUUGGUUUACGGUAGGCGCCGG
CGGGGGAGACAUUUAUCACAGCAUGUCUCAUGCCCGACCCCGCUA
SEQ ID NO: 19
(FIG. 8F - HCV 5 NS5B region)
GCUGACGCCGAUCGGCUGGACUUGUCCAGCUGGUUUACCGUUGGCGCCGG
CGGGGGGGACAUUUAUCACAGCAUGUCCCGUGCCCGACCCCGCUG
SEQ ID NO: 20
(FIG. 8G - HCV 6 NS5B region)
GGUCUCCGCGAGCAAGCUUGACUUAUCAGGCUGGUUCGUGGCAGGCUACG
ACGGGGGGGACAUUUAUCACAGCGUGUCCCAGGCCCGACCCCGUU
SEQ ID NO: 21
primer
AGGCGATTAAGTTGGGTA
SEQ ID NO: 22
primer
CGGTAGGCGTGTACGGTG
SEQ ID NO: 23
primer
TAG GAG GGC CCA TGT TCA AC
SEQ ID NO: 24
primer
CCC CTG GCT TTC TGA GAT GAC
SEQ ID NO: 25
primer
TAA TGA AGG ACT TGG CAG ATG AAC T
SEQ ID NO: 26
primer
ACG GCT TTC TCC CTC TTG CT
SEQ ID NO: 27
primer
CTG TCT TCA CGC AGA AAG CG
SEQ ID NO: 28
primer
CAC TCG CAA GCA CCC TAT CA
SEQ ID NO: 29
(from FIG. 2)
GGCGGGGCGCGGGC
SEQ ID NO: 30
(from FIG. 2)
GUCCGCGCCUCGCC
SEQ ID NO: 31
(from FIG. 2)
GCGCCG
SEQ ID NO: 32
(from FIG. 2)
CCGG
SEQ ID NO: 33
(from FIG. 2)
CCGG
SEQ ID NO: 34
(from FIG. 2)
GGGCGC

What is claimed is:

1. A composition comprising an isolated nucleic acid sequence that has a similar or identical secondary structure to a native NS5B, X, BA, or EC region of a hepatitis C viral RNA.

2. The composition of claim 1, wherein the isolated nucleic acid sequence has a similar or identical secondary structure to the native NS5B region of the hepatitis C viral RNA, wherein the nucleic acid sequence does not have significant sequence identity to the NS5B region, and wherein the isolated nucleic acid sequence binds to or inhibits the activity of NS5B polymerase.

3. The composition of claim 1, wherein the isolated nucleic acid sequence has a length of about 50 to about 150 bases.

4. The composition of claim 1, wherein the isolated nucleic acid sequence differs from the native NS5B, X, BA, or EC region in a sequence which forms a stem of a stem-loop secondary structure.

5. The composition of claim 1, further comprising a pharmaceutically acceptable carrier.

6. The composition of claim 1, wherein the isolated nucleic acid sequence is selected from the group consisting of SEQ ID NO:3, SEQ ID NO:2, SEQ ID NO:5, SEQ ID NO:9, SEQ ID NO:14, SEQ ID NO:15, SEQ ID NO:16, SEQ ID NO:17, SEQ ID NO:18, SEQ ID NO:19, and SEQ ID NO:20.

7. The composition of claim 1, wherein the isolated nucleic acid sequence has between about 45% and about 95% sequence identity to a sequence selected from the group consisting of SEQ ID NO:1, SEQ ID NO:2, SEQ ID NO:4, SEQ ID NO:6, SEQ ID NO:8, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar or identical secondary structure to SEQ ID NO:1, SEQ ID NO:2, SEQ ID NO:4, SEQ ID NO:6, SEQ ID NO:8, SEQ ID NO:14, SEQ ID NO:15, SEQ ID NO:16, SEQ ID NO:17, SEQ ID NO:18, SEQ ID NO:19, or SEQ ID NO:20.

8. The composition of claim 1, wherein the isolated nucleic acid sequence has between about 45% and 73% sequence identity to a sequence selected from the group consisting of SEQ ID NO:3, SEQ ID NO:5, and SEQ ID NO:9, wherein the isolated sequence has a similar or identical secondary structure to SEQ ID NO:3, SEQ ID NO:5, or SEQ ID NO:9.

9. The composition of claim 1, wherein the isolated nucleic acid sequence has between about 75% and 95% sequence identity to a sequence selected from the group consisting of SEQ ID NO:3, SEQ ID NO:5, and SEQ ID NO:9, wherein the isolated sequence has a similar or identical secondary structure to SEQ ID NO:3, SEQ ID NO:5, or SEQ ID NO:9.

10. The composition of claim 1, comprising at least two different isolated nucleic acid sequences, wherein each isolated nucleic acid sequence has a similar or identical secondary structure to a native NS5B, X, BA, or EC region of a hepatitis C viral RNA.

11. A method for inhibiting replication of a hepatitis C virus, comprising:
adding a composition to the hepatitis C virus, wherein the composition comprises an isolated nucleic acid sequence that has a similar or identical secondary structure to a native NS5B, X, BA, or EC region of a hepatitis C viral RNA; and
inhibiting replication of the hepatitis C virus.

12. The method of claim 11, wherein the isolated nucleic acid sequence has a similar or identical secondary structure to the native NS5B region of the hepatitis C viral RNA, and wherein the nucleic acid sequence does not have significant sequence identity to the NS5B region, and wherein the isolated nucleic acid sequence binds to or inhibits the activity of NS5B polymerase.

13. The method of claim 11, wherein the hepatitis C virus has a genotype selected from the group consisting of 1b, 2a, 1a, 2b, 2c, or 3a.

14. The method of claim 11, wherein the composition comprises an isolated nucleic acid sequence having at least about 45% sequence identity to a sequence selected from the group consisting of SEQ ID NO:1, SEQ ID NO:2, SEQ ID NO:3, SEQ ID NO:4, SEQ ID NO:5, SEQ ID NO:8, SEQ ID NO:9, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar or identical secondary structure to SEQ ID NO:1, SEQ ID NO:3, SEQ ID NO:4, SEQ ID NO:5, SEQ ID NO:8, SEQ ID NO:9, SEQ ID NO:14, SEQ ID NO:15, SEQ ID NO:16, SEQ ID NO:17, SEQ ID NO:18, SEQ ID NO:19, or SEQ ID NO:20.

15. A method for treating a patient having a hepatitis C virus, comprising:
administering to the patient a composition comprising a therapeutically effective amount of an isolated nucleic acid sequence that has a similar or identical secondary structure to a native NS5B, X, BA, or EC region of a hepatitis C viral RNA, and a pharmaceutically acceptable carrier.

16. The method of claim 15, wherein the isolated nucleic acid sequence has a similar or identical secondary structure to the native NS5B region of the hepatitis C viral RNA, wherein the nucleic acid sequence does not have significant sequence identity to the NS5B region, and wherein the isolated nucleic acid sequence binds to or inhibits the activity of NS5B polymerase.

17. The method of claim 15, wherein the patient has hepatitis C virus genotype 1b or 2a.

18. The method of claim 15, wherein the patient has hepatitis C virus particles of two or more genotypes or subtypes.

19. The method of claim 15, further comprising administering to the patient a pegylated interferon or ribavirin.

20. The method of claim 15, wherein the composition comprises a therapeutically effective amount of an isolated nucleic acid sequence having at least about 45% sequence identity to a sequence selected from the group consisting of SEQ ID NO:1, SEQ ID NO:2, SEQ ID NO:3, SEQ ID NO:4, SEQ ID NO:5, SEQ ID NO:8, SEQ ID NO:9, SEQ ID NO:14, SEQ ID NO:15, SEQ ID NO:16, SEQ ID NO:17, SEQ ID NO:18, SEQ ID NO:19, and SEQ ID NO:20, wherein the isolated nucleic acid sequence has a similar secondary structure to SEQ ID NO:1, SEQ ID NO:2, SEQ ID NO:3, SEQ ID NO:4, SEQ ID NO:5, SEQ ID NO:8, SEQ ID NO:9, SEQ ID NO:14, SEQ ID NO:15, SEQ ID NO:16, SEQ ID NO:17, SEQ ID NO:18, SEQ ID NO:19, or SEQ ID NO:20.

21. The method of claim 15, wherein the composition comprises at least two different isolated nucleic acid sequences, wherein each isolated nucleic acid sequence has a similar or identical secondary structure to a native NS5B, X, BA, or EC region of a hepatitis C viral RNA.

22. A method for identifying a nucleic acid sequence that inhibits viral replication, comprising:
selecting a target sequence on a native viral nucleic acid, wherein the presence of additional copies of such target sequence affects viral replication;
using a computer to predict the secondary or tertiary structure of the target sequence; and
using the computer to identify a nucleic acid sequence that has a similar or identical predicted secondary or tertiary structure to the target sequence, wherein the nucleic acid that has a similar or identical predicted secondary or tertiary structure to the target sequence inhibits viral replication.

23. The method of claim 22, wherein the native viral nucleic acid is a hepatitis C viral (HCV) RNA.

24. The method of claim 22, wherein the target sequence is a native NS5B, X, BA, or EC region of the HCV RNA.

25. The method of claim 23, wherein the computer program predicts secondary structure and the identified nucleic acid sequence does not have significant sequence similarity to the target sequence.

26. The method of claim 23, wherein the target sequence is a native NS5B region of HCV RNA, and wherein the identified nucleic acid sequence binds to or inhibits the activity of NS5B polymerase.


SEQUENCE LISTING (34 sequences)

SEQ ID NO: 1 - RNA, 95 nt, Artificial Sequence
  Hepatitis C Virus
ccggcugcgucccaguuggauuuauccagcugguucguugcugguuacagcgggggagacauauaucacagccugucucgugcccgaccccgcug

SEQ ID NO: 2 - RNA, 95 nt, Artificial Sequence
  Hepatitis C Virus
cggcggggcgcgggcuucgauuuaucgagguccgcgccucgccguuacgcgccgggccggauauaucacagccuccggcgugcccgacgggcgcg

SEQ ID NO: 3 - RNA, 95 nt, Artificial Sequence
  Hepatitis C virus
ccggaggcgcgccuacuggacuuauccaguugguucaccgucggcgccggcgggggcgacauuuuucacagcgugucgcgcgcccgaccccgcuc

SEQ ID NO: 4 - RNA, 98 nt, Artificial Sequence
  Hepatitis C Virus
gguggcuccaucuuagcccuagucacggcuagcugugaaagguccgugagccgcuugacugcagagagugcugauacuggccucucugcagaucaagu

SEQ ID NO: 5 - DNA, 95 bp, Artificial Sequence
  Hepatitis C Virus
ggtggctccatcttagccctagtcacggctagctgtgaaaggtccgtgagccgcatgactgcagagagtgccgtaactggtctctctgcagatca

SEQ ID NO: 6 - RNA, 116 nt, Artificial Sequence
  Hepatitis C Virus
ccaggcauugagcggguugauccaagaaaggacccggucguccuggcaauuccgguguacucaccgguuccgcagaccacuauggcucucccgggaggggggguccuggaggcugc

SEQ ID NO: 7 - DNA, 116 bp, Artificial Sequence
  Hepatitis C Virus
ccgggcatagagtgggtttatccaagaaaggacccagtcttcccggcaattccggtgtactcaccggttccgcagaccactatggctctcccgggaggggggggcctggaggctgt

SEQ ID NO: 8 - RNA, 104 nt, Artificial Sequence
  Hepatitis C Virus
gacacucauacuaacgccauggcuagacgcuuucugcgugaagacaguaguuccucacaggggagugaucuaugguggagugucgcccccaaucgggggcuggc

SEQ ID NO: 9 - DNA, 104 bp, Artificial Sequence
  Hepatitis C Virus
cgacactcatactaacgccatggctaggcgctttctgcgtgaagacagtagttcctcacaggggagtgattcatggcggagtgtcgcccctattaggggcaggt

SEQ ID NO: 10 - RNA, 21 nt, Artificial Sequence
  Synthetic construct
ccggcugcgucccaguuggau

SEQ ID NO: 11 - RNA, 22 nt, Artificial Sequence
  Synthetic construct
uuauccagcugguucguugcug

SEQ ID NO: 12 - RNA, 28 nt, Artificial Sequence
  Synthetic construct
guuacagcgggggagacauauaucacag

SEQ ID NO: 13 - RNA, 24 nt, Artificial Sequence
  Synthetic construct
ccugucucgugcccgaccccgcug

SEQ ID NO: 14 - DNA, 94 bp, Artificial Sequence
  Hepatitis C virus
ggccgctagcccagctggacttgtccggttggttcacggctggctacagcgggggagacatttatcacagcgtgtctcgtgcccggccccgctg

SEQ ID NO: 15 - RNA, 95 nt, Artificial Sequence
  Hepatitis C virus
cccgaggcgagccgccuagauuuauccgggugguucaccgugggcgccggcgggggcgacaucuuucacagcgugucgcaugcccgaccccgccu

SEQ ID NO: 16 - RNA, 95 nt, Artificial Sequence
  Hepatitis C virus
ccggcggcacgccuccuggacuuguccagcugguucaccgucagcgcuggcgggggcgacauauaucacagcgugucgcgagcucggccccgccu

SEQ ID NO: 17 - RNA, 95 nt, Artificial Sequence
  Hepatitis C virus
ccagccgcuggccaguuggauuuauccagcugguuuacgguuggcgucggcgggaacgacauuuaucacagcgugucacgugcccgaacccgcua

SEQ ID NO: 18 - RNA, 95 nt, Artificial Sequence
  Hepatitis C virus
ccugccgcugccaaacucgauuuaucggguugguuuacgguaggcgccggcgggggagacauuuaucacagcaugucucaugcccgaccccgcua

SEQ ID NO: 19 - RNA, 95 nt, Artificial Sequence
  Hepatitis C virus
gcugacgccgaucggcuggacuuguccagcugguuuaccguuggcgccggcgggggggacauuuaucacagcaugucccgugcccgaccccgcug

SEQ ID NO: 20 - RNA, 95 nt, Artificial Sequence
  Hepatitis C virus
ggucuccgcgagcaagcuugacuuaucaggcugguucguggcaggcuacgacgggggggacauuuaucacagcgugucccaggcccgaccccguu

SEQ ID NO: 21 - DNA, 18 bp, Artificial Sequence
  Synthetic construct
aggcgattaagttgggta

SEQ ID NO: 22 - DNA, 18 bp, Artificial Sequence
  Synthetic construct
cggtaggcgtgtacggtg

SEQ ID NO: 23 - DNA, 20 bp, Artificial Sequence
  Synthetic construct
taggagggcccatgttcaac

SEQ ID NO: 24 - DNA, 21 bp, Artificial Sequence
  Synthetic construct
cccctggctttctgagatgac

SEQ ID NO: 25 - DNA, 25 bp, Artificial Sequence
  Synthetic construct
taatgaaggacttggcagatgaact

SEQ ID NO: 26 - DNA, 20 bp, Artificial Sequence
  Synthetic construct
acggctttctccctcttgct

SEQ ID NO: 27 - DNA, 20 bp, Artificial Sequence
  Synthetic construct
ctgtcttcacgcagaaagcg

SEQ ID NO: 28 - DNA, 20 bp, Artificial Sequence
  Synthetic construct
cactcgcaagcaccctatca

SEQ ID NO: 29 - RNA, 14 nt, Artificial Sequence
  Synthetic construct
ggcggggcgcgggc

SEQ ID NO: 30 - RNA, 14 nt, Artificial Sequence
  Synthetic construct
guccgcgccucgcc

SEQ ID NO: 31 - RNA, 6 nt, Artificial Sequence
  Hepatitis C virus
gcgccg

SEQ ID NO: 32 - RNA, 4 nt, Artificial Sequence
  Hepatitis C virus
ccgg

SEQ ID NO: 33 - RNA, 4 nt, Artificial Sequence
  Hepatitis C virus
ccgg

SEQ ID NO: 34 - RNA, 6 nt, Artificial Sequence
  Hepatitis C virus
gggcgc